(19) United States
(12) Patent Application Publication
(10) Pub. No.: US 2006/0015181 A1
(43) Pub. Date: Jan. 19, 2006

(54) Interspinous vertebral implant

(75) Inventor: Jean-Francois Elberg, Neuilly Sur Seine (FR)

Correspondence Address: BURR & BROWN, PO BOX 7068, SYRACUSE, NY, 13261-7068, US

(73) Assignee: Biomet Merck France (50% interest), Valence (FR)

(21) Appl. No.: 10/894,285

(22) Filed: Jul. 19, 2004

(51) Int. Cl. A61F 2/28

(52) U.S. Cl. 623/16.11

(57) ABSTRACT

This interspinous vertebral implant comprises a body (11) which is intended to be inserted between two consecutive interspinous processes (3, 4), is compressible in the direction of the spine and is provided with means for anchoring it to at least one spinous process.

The body (11) is formed by a single loop closed upon itself.

The anchoring means are formed by two fixing lugs (12, 13) which are integral with the body (11), extend on either side of the spinous process in the area of which they are intended to anchor when the implant is in place, and are each traversed by a through-opening (14, 15) oriented in a direction substantially perpendicular to the general plane of said process, the through-openings being intended to receive fixing studs (23) to be crimped in the area of the process in question.

The invention concerns an interspinous vertebral implant intended to function as a stabilizer between two consecutive vertebrae. More precisely, such an implant is intended to be positioned between the spinous processes of two consecutive vertebrae.

The invention is applied directly in the context of the phenomena known as vertebral destabilization. These phenomena are manifested by abnormal movements of the vertebral column, more particularly in the lumbar region, and result in pain called lumbago.

Although some forms of lumbago can be treated by physiotherapy, other forms, by contrast, are of a more permanent nature and drastically incapacitate the affected patient. These forms of lumbago can also result from damage or degeneration of the intervertebral disc, which can lead to abnormal play of the vertebrae contiguous to this disc.

In order to overcome these problems of instability, it was first proposed to perform arthrodesis, that is to say mechanical immobilization of the consecutive vertebrae concerned. To do this, rigid elements, in most cases consisting of metal rods, are implanted along several vertebrae on either side of the spinous processes. The bone anchoring means of these rods generally consist of so-called pedicle screws implanted in the area of the pedicles of the vertebrae.

This type of device in the first place has the disadvantage of being relatively difficult to implant, requiring extensive and complex work to fit it.

In addition, it immobilizes a relatively long vertebral segment, which significantly reduces the patient's mobility and can subject the vertebral articulations situated either side of this rigid segment to considerable stresses, which may cause new instability in this area.

To overcome these problems of instability, it has also been proposed to fit ligaments, these also being implanted in the area of pedicle screws.

Unfortunately, this has posed the problem of the relative fragility of these ligaments, and in addition the fact that they work only in the direction of extension, not of compression. For this reason, they do not provide an effective and lasting solution to the aforementioned instabilities.

It was then proposed to insert a wedge between the spinous processes of the vertebrae concerned. Such a wedge is fixed in this area using tissue ligaments, for example made of Dacron (trademark), surrounding the adjacent processes.

In addition to the complexity of fitting such a wedge, the need to pass the ligament around the processes involves an intervention on healthy areas in order to form a passage and then weaken the natural ligaments.

It has also been proposed, for example in document WO 99/40866, to use an interspinous stabilizer comprising a body which is compressible in the direction of the spine, is intended to be inserted between two consecutive spinous processes and is provided with members for anchoring it to the spinous processes of the two respective vertebrae.

Although this implant does indeed make it possible to remedy the various aforementioned disadvantages while at the same time ensuring the desired stabilization, experience by contrast shows that, in the event of flexion of the column, especially in the lumbar region, said implant is susceptible to being expelled from its site of implantation: in other words its fixation in the interspinous zone is not sufficient to avoid this type of pitfall.

The subject of the invention is an interspinous implant which is able to permit at least local stabilization of the spine, while avoiding all of the above disadvantages.

This interspinous vertebral implant comprises a body which is intended to be inserted between two consecutive interspinous processes, is compressible in the direction of the spine and is provided with means for anchoring it to at least one spinous process.

It is characterized in that the body is formed by a single loop closed upon itself, in that said anchoring means consist of two fixing lugs which are integral with the body, extend on either side of the spinous process in the area of which they are intended to anchor when the implant is in place, and are each traversed by through-openings, in a direction substantially perpendicular to the general plane of said processes, the through-openings being intended to receive fixing studs to be crimped in the area of the process in question.

This being the case, the intervertebral stabilizer implant according to the invention permits a certain degree of mutual mobility of two vertebrae in the area of which it is implanted, thus partially reproducing the biomechanics of a healthy intervertebral disc.

Such an implant additionally permits flexion or extension of the spine, and its use does not require an invasive surgical procedure.

According to an advantageous characteristic of the invention, the intervertebral body is formed by a spring blade closed upon itself, substantially in the shape of a figure of 8 or kidney bean, and symmetrical with respect to the median plane.

The anchoring members are formed by fixing lugs, also mutually symmetrical with respect to the aforementioned median plane, even parallel to one another, said fixing lugs having, near their free end, a through-opening of truncated cone shape functioning as a morse cone able to cooperate with anchoring studs of complementary shape, thus ensuring their retention in this area once the studs have been crimped in the spinous process.

According to a first embodiment of the invention, the interspinous body comprises two sets of two fixing lugs in order to thus permit fixation of the interspinous vertebral implant in the area of the corresponding processes of two consecutive vertebrae.

According to another variant of the invention, the fixing members comprise, on the one hand, two fixing lugs in the area of an interspinous process, and, on the other hand, an arc which diverges from said body and whose free ends each comprise a through-opening which permits passage, in this area, of pedicle screws or osteosynthesis screws.

The manner in which the invention can be realized, and the advantages which it affords, will become clearer from the following embodiments which are given by way of non-limiting examples and with reference to the attached figures.

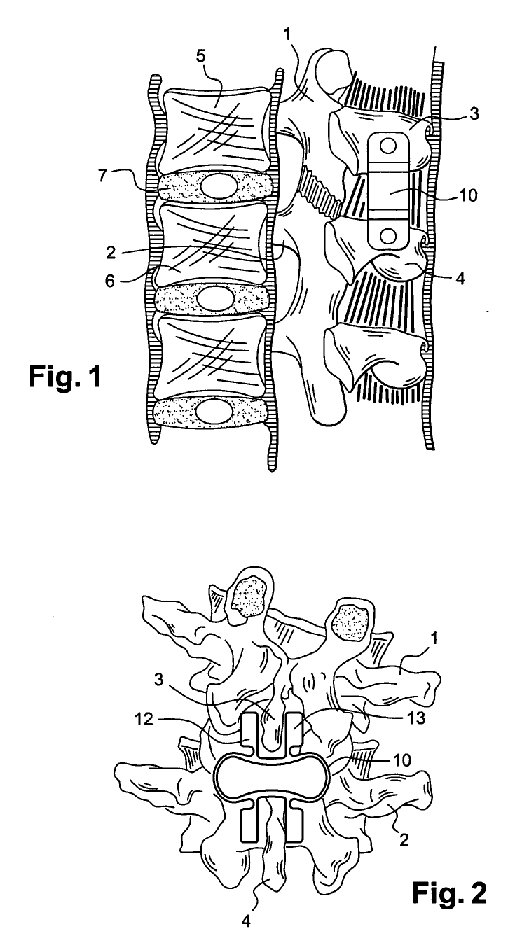

FIG. 1 is a diagrammatic partial perspective view of the device according to the invention, fitted in the area of two consecutive lumbar vertebrae.

FIG. 2 is a diagrammatic view, in partial cross section, of FIG. 1.

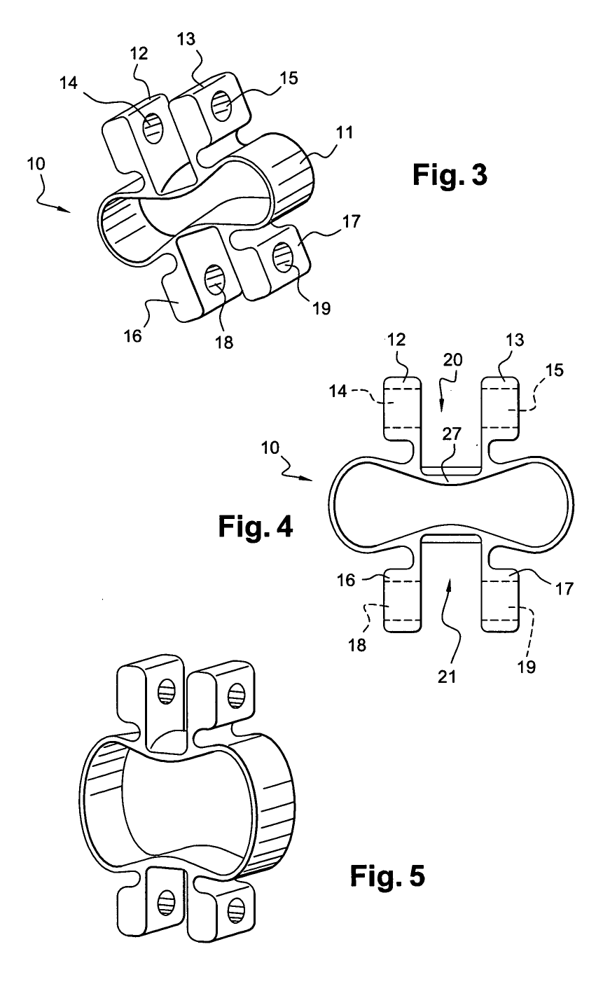

FIG. 3 is a diagrammatic perspective view of the interspinous vertebral implant according to a first embodiment of the invention.

FIG. 4 is a diagrammatic view, in cross section, of the interspinous vertebral implant from FIG. 3.

FIG. 5 is a view similar to FIG. 4, in which the implant has different dimensions.

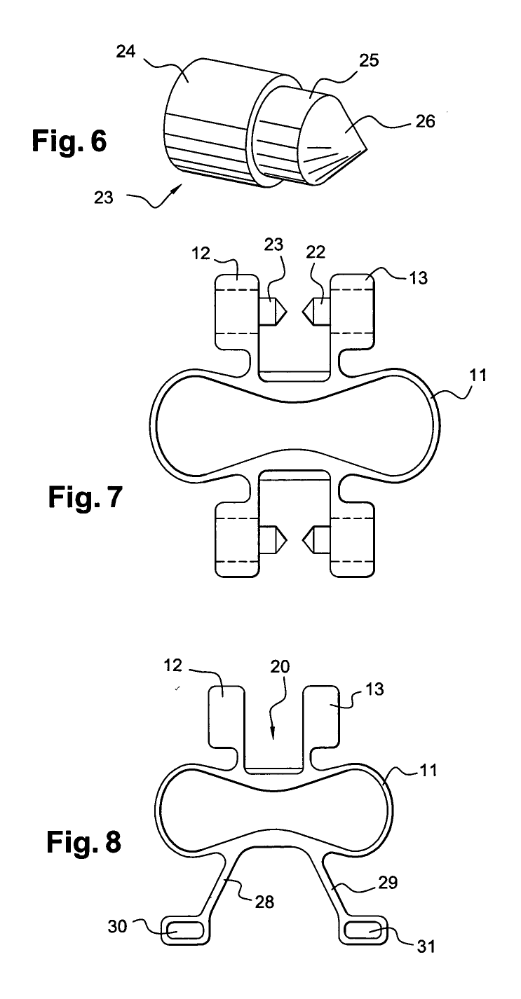

FIG. 6 is a diagrammatic perspective view of a fixing stud of the implant according to the invention.

FIG. 7 is a diagrammatic front view of the implant according to the invention, provided with fixing studs.

FIG. 8 is a diagrammatic front view of the implant according to another embodiment of the invention.

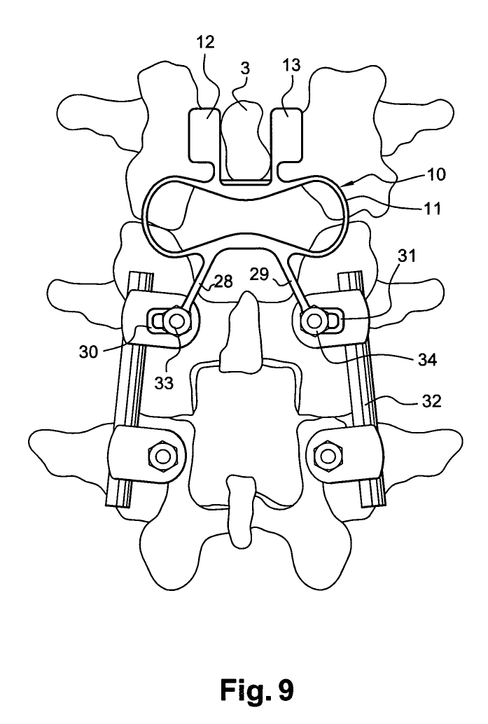

FIG. 9 is a diagrammatic view illustrating the use of the implant from FIG. 8.

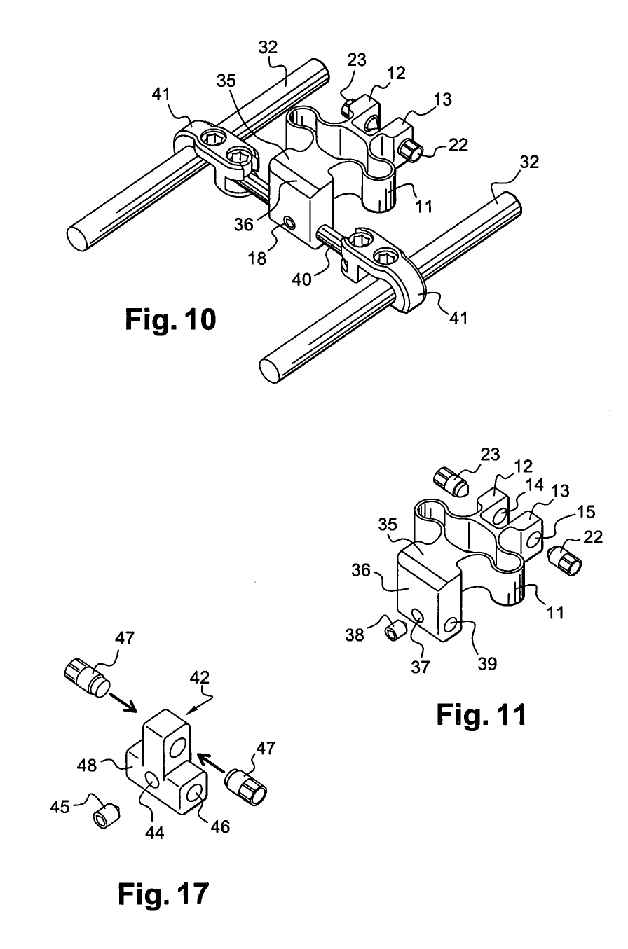

FIGS. 10 and 11 are diagrammatic perspective views illustrating another embodiment of the invention, constituting a variant of FIG. 9, and using a one-piece implant which can be fixed to the end of a lumbar arthrodesis.

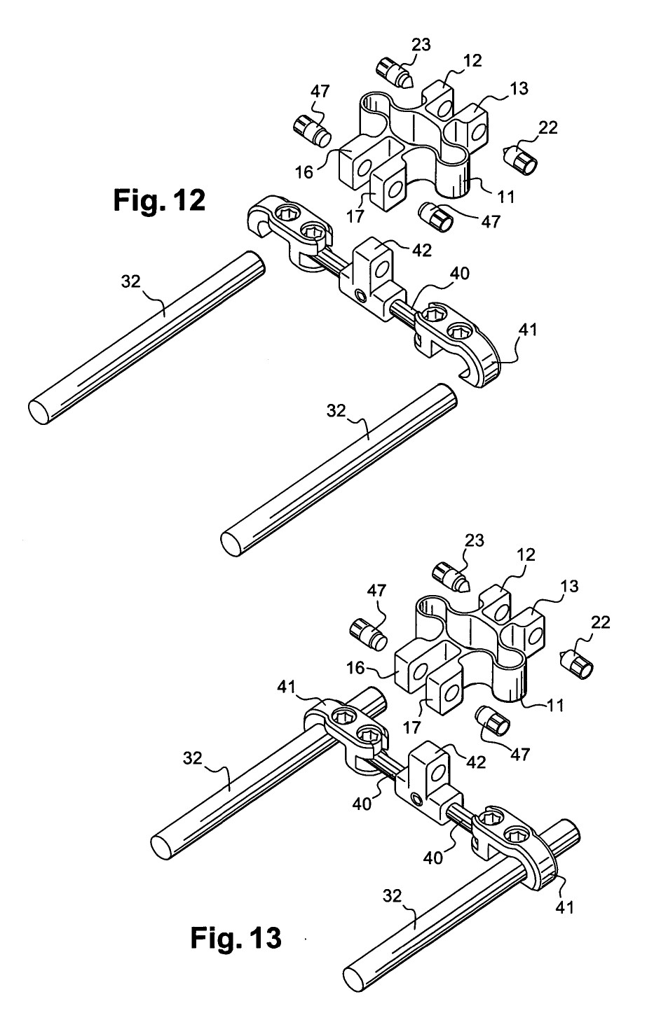

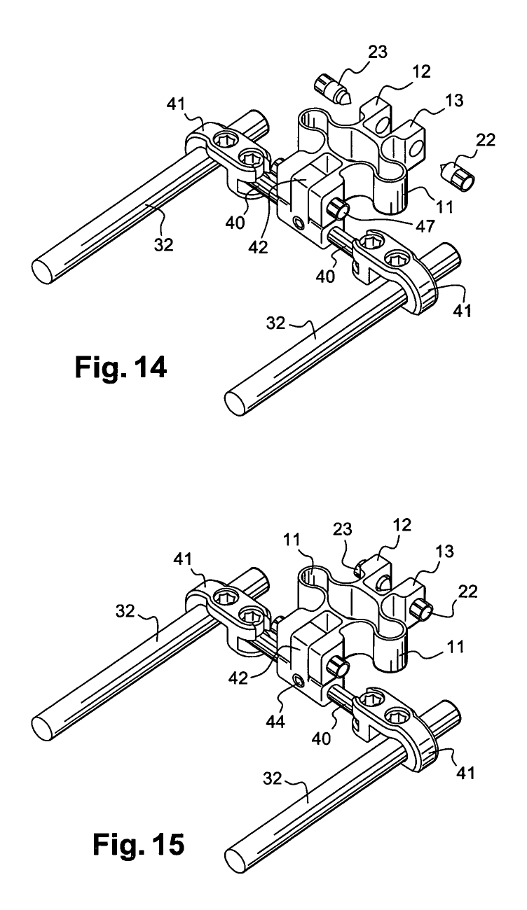

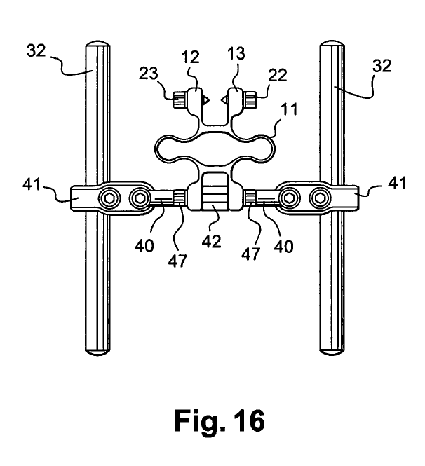

FIGS. 12 to 15 illustrate, in exploded views, a junction implant intended to form the transition between the interspinous implant proper and a lumbar arthrodesis, FIG. 16 being a plan view, and FIG. 17 being a diagrammatic view of one of the connection elements.

In FIGS. 1 and 2, the interspinous vertebral implant according to the invention has been shown fitted in the area of two consecutive lumbar vertebrae. These lumbar vertebrae bear general reference numbers (1) and (2), in the area of which are illustrated, in particular, the respective spinous processes (3) and (4), the respective vertebral bodies (5) and (6), and an intervertebral disc (7).

As can clearly be seen from these FIGS. 1 and 2, the implant (10) of the invention is intended to be inserted between two consecutive spinous processes (3) and (4). This implant is described more particularly with reference to FIGS. 3 and 4.

It is basically made up of a body (11) formed by a spring blade closed upon itself, in the shape of a figure of 8 open at its centre, or in the shape of a kidney bean. This spring blade thus gives the implant a certain degree of compressibility, which degree can be varied as a function of the thickness given to the spring blade.

Given the particular configuration of the body and its placement in the area of the lumbar spine, the direction of the compressibility is substantially aligned with that of the spine, specifically in such a way as to permit both the movements of compression and of extension of the spine when the implant is positioned between the spinous processes.

This body (11) is symmetrical with respect to the vertical median plane. It is advantageously made of titanium or of a titanium alloy of type TA6V, that is to say a titanium alloy comprising 6% by weight of aluminium and 4% by weight of vanadium.

On either side of its two main faces, the body has anchoring lugs (12), (13) and (14), (15), respectively, the two lugs of each of the sets being symmetrical with respect to the aforementioned median plane. The shape of the body can be adapted depending on the site of implantation of the intervertebral stabilizer, by acting on its particular configuration, in particular still in the shape of a figure of 8 or kidney bean, and on its dimensions, for example as can be observed in FIG. 5.

These anchoring lugs in this case extend in two directions parallel to one another and define a respective free space (20, 21) permitting passage, in this area, of the spinous processes of the vertebrae in question, as can be observed in FIG. 1. The two lugs (12, 13) of the same anchoring member thus extend opposite one another. Each of these two lugs has, near its free end, a through-opening (14) and (15), respectively, in the form of a morse cone.

These through-openings are oriented substantially perpendicular to the general plane containing the spinous process, as can be discerned for example from FIG. 2. The same applies to the lugs (16) and (17) provided with through-openings (18) and (19), respectively, also in the form of a morse cone.

The whole implant is in one piece and is forged from a single blank based on a titanium alloy, as has been mentioned above.

The openings (14), (15), (18) and (19) are each intended to cooperate with a fixing stud (23), as is shown in FIG. 6.

This fixing stud thus first comprises an insertion zone (24), also in the form of a morse cone and with a shape complementing the internal shape of the through-openings (14, 15, 18 and 19). This portion in the form of a morse cone (24) is continued by a cylindrical zone (25), of smaller diameter, which ends in a pointed profile (26) in order to permit crimping of said stud in the spinous process and, by this means, fixation of the implant.

In fact, when the studs are inserted into the aforementioned openings, only the zones (25) and (26) emerge into the space (20), (21) between the lugs, as can be observed from FIG. 7.

This implant thus has an anatomical profile and proves to provide a certain degree of comfort for the patient. In order to optimize this anatomical profile, the implant has a certain excess thickness (27) near the zones of connection of the lugs (12, 13, 16, 17) to the body (11).

The implant according to the invention can be inserted by simple engagement in the interspinous space between the two adjacent vertebrae in question, requiring only a limited intervention both in terms of invasiveness and time, since it suffices to spread the spinous processes slightly apart to permit its placement with the aid of a suitable ancillary means.

In addition, such an implant does not suffer any wear, since it is not subjected to repeated friction. In this way, it can be maintained in place for many years, without needing replacement.

Moreover, given its principle of fixation, it is possible to retain the relative mobility of the two vertebrae concerned, both in terms of compression and extension, but also the relative movement of the vertebrae with respect to one another in a direction perpendicular to the axis of the spine, given that the method of fixation by studs functions as it were as a hinge, authorizing such relative displacement.

Another embodiment of the invention is shown in FIGS. 8 and 9. Essentially, the body (11) is basically identical to the one illustrated in the previous figures but has only two lugs (12), (13) anchoring it on the spinous process of a lumbar vertebra.

By contrast, the lugs (16) and (17) are replaced by two lugs (28, 29) which are substantially in the shape of a divergent arc and whose two free ends are provided with a through-opening (30, 31) intended to permit insertion of fixing screws at this level. These through-openings are no longer oriented perpendicular to the general plane of the spinous processes, but parallel to this plane.

This embodiment is intended, for example, for the specific area of the junction between the lumbar vertebrae and the sacrum, and in particular between the lumbar vertebra L5 and the first vertebra S1 of the sacrum. In this area, in fact, the vertebra S1 has no spinous process, so that, in order to permit stabilization of the spine in this area, it is necessary to provide another method of fixing, such as by means of the aforementioned divergent arc.

This divergent arc can additionally have a much larger segment to permit implantation of the interspinous vertebral implant between a zone of the spine presenting a fixation system of the arthrodesis type, hence rigid, and an underlying zone. Thus, FIG. 9 shows this implant fitted between a lumbar vertebra and an arthrodesis (32). According to this configuration, the openings (30, 31) situated respectively at the end of the lugs (28) and (29) engage on the respective pedicle screws (33, 34) of the upper zone of the arthrodesis and are maintained at this level by means of a nut or equivalent system.

In other words, the openings (30, 31) with which the two free ends of the arc are provided are intended to receive the pedicle screws (33, 34) of the arthrodesis in question, provided for this purpose with a free end equipped with a suitable thread.

This being the case, the implant makes it possible to combat the junction syndrome which is familiar when using arthrodesis systems.

A variant of this particular design of the implant is shown in FIGS. 10 and 11. According to this variant, the implant is still intended to be fixed to the end of a lumbar arthrodesis, but it is of a one-piece structure.

In other words, this time it is without the two lugs (28, 29) forming the divergent arc, and, instead, it has in place of these a block (35) extending from the lower part of the body (11) of the implant. This block (35) has, in the area of its base (36), a bore (39) which passes right through it in such a way as to receive, in this area, a rod or bar (40) of suitable diameter. In a known manner, this rod or bar (40) is capable of receiving, at its two free ends, means (41) for connection to the arthrodesis rods (32). These means are familiar to the person skilled in the art, so that there is no need to describe them in any detail here.

The rod or bar (40) is made integral with the base (36) of the block (35) by tightening a screw (38) in a bore (37) which opens into the bore (39). This screw (38) is, for example, of the type with a hexagonal head or star-shaped head, such as are sold under the trademark TORX®.

In the example described, the bore (39) receiving the rod or bar (40) for connection to the arthrodesis system is formed in the posterior zone of the base (36) of the block (35), in order to take account of the offset of the planes receiving, respectively, the implant and the arthrodesis rods.

In another variant illustrated in FIGS. 12 to 17 and intended to afford greater latitude to the practitioner when fitting the implant of the invention, the block (35) is replaced by an intermediate connection element (42) which is received between the lower lugs (16, 17) of the standard implant of the invention and for this reason constitutes intermediate fixing lugs. To this end, it has, in its upper part, two bores (43) intended to cooperate with immobilizing studs or screws (47) of the type with a hexagonal head or head of the TORX® type, after introduction of said screws into the openings (18, 19) with which the lower lugs (16, 17) of the implant are provided.

The base (48) of the intermediate connection element (42) is, like the block (35), provided with a through-bore (46) able to receive an immobilizing rod or bar (40). In the same way, the base (48) is also provided with an opening (44) which opens into the through-bore (46) and is intended to receive a screw (45) for immobilizing the aforementioned rod or bar (40) in the desired position.

With this configuration, it will be appreciated that it thus becomes easier for the practitioner to adapt the implant according to the invention to an existing arthrodesis, and, consequently, this greatly limits the intervention proper. In addition, this particular configuration of the invention makes it possible to relieve the intervertebral discs lying above and/or below a lumbar arthrodesis.

Thus, all the benefits of the present invention will be appreciated, namely that the vertebral implant ensures local stabilization of the spine in the area of its site of implantation, while at the same time making it possible to perform very rapid and minimally invasive surgery, and additionally avoiding any risk of expulsion of the implant during the various movements of the spine, especially during flexion movements.

1. Interspinous vertebral implant comprising a body (11) which is intended to be inserted between two consecutive interspinous processes (3,4), is compressible in the direction of the spine and is provided with means for anchoring it to at least one spinous process,
wherein the body (11) is formed by a single loop closed upon itself,
wherein said anchoring means consist of two fixing lugs (12, 13) which are integral with the body (11), extend on either side of the spinous process in the area of which they are intended to anchor when the implant is in place, and are each traversed by a through-opening (14, 15) oriented in a direction substantially perpendicular to the general plane of said process, the through-openings being intended to receive fixing studs (23) to be crimped in the area of the process in question;
and wherein said fixing studs (23) thus first comprise an insertion zone (24) in the form of a morse cone and with a shape complementing the internal shape of the through-openings (14, 15), this zone in the form of a morse cone (24) being continued by a cylindrical zone (25), of smaller diameter, which ends in a pointed profile (26) in order to permit crimping of said stud in the spinous process and, by this means, fixation of the implant.

2. Interspinous vertebral implant according to claim 1, wherein the body (11) is formed by a spring blade closed upon itself, substantially in the shape of a figure of 8 or kidney bean, and symmetrical with respect to the median plane.

3. Interspinous vertebral implant according to claim 1, wherein the anchoring means are formed by fixing lugs (12, 13, 16, 17), also mutually symmetrical with respect to the median plane of the body (11), even parallel to one another, said fixing lugs having, near their free end, a through-opening (14, 15, 18, 19) of truncated cone shape functioning as a morse cone able to cooperate with anchoring studs (23) of complementary shape, thus ensuring their retention in this area once the studs have been crimped in the spinous process.

4. Interspinous vertebral implant according to claim 1, wherein the body (11) comprises two sets of two fixing lugs in order to thus permit fixation of the interspinous vertebral implant in the area of the corresponding processes of two consecutive vertebrae.

5. Interspinous vertebral implant according to claim 1, wherein the body (11) comprises a first set of two fixing lugs (12, 13) on a spinous process, and a second set of fixing lugs (28, 29) which are substantially in the form of a divergent arc and whose two free ends are each provided with a through-opening (30, 31) intended to permit insertion of fixing screws (33, 34) in this area.

6. Interspinous vertebral implant according to claim 5, wherein the through-openings (30, 31) are oriented parallel to the general plane of the spinous processes.

7. Interspinous vertebral implant according to claim 1, wherein the body (11) comprises a first set of two fixing lugs (12, 13) on a spinous process, and a block (35) extending from the opposite face of said body, said block (35) having, in the area of its base (36), a through-bore (39) intended to receive a rod or bar (40) for fixing on an arthrodesis system (32).

8. Interspinous vertebral implant according to claim 7, wherein the fixing rod or bar (40) is made integral with the base (36) of the block (35) by tightening a screw (38) in a bore (37) which opens into the bore (39).

9. Interspinous vertebral implant according to claim 1, wherein the body (11) comprises a first set of two fixing lugs (12, 13) on a spinous process, and a second set of two intermediate fixing lugs (14, 15), said second set receiving an intermediate connection element (42) provided, in its upper part, with two bores (43) which are intended to cooperate with immobilizing studs or screws (47) after introduction of said screws into the orifices (18, 19) with which the lower lugs (16, 17) of the implant are provided, and whose base (48) is provided with a through-bore (46) intended to receive a rod or bar (40) for fixing on an arthrodesis system (32).

10. Interspinous vertebral implant according to claim 9, wherein the fixing rod or bar (40) is made integral with the base (48) of the intermediate connection element (42) by tightening of a screw (45) in a bore or orifice (44) which opens into the through-bore (46).
